(19) United States
(12) Patent Application Publication
(10) Pub. No.: US 2018/0364169 A1
(43) Pub. Date: Dec. 20, 2018

(54) NANOPORE-BASED ANALYSIS OF COMPOUNDS USING MOBILE FRET PAIRS

(71) Applicant: Quantapore, Inc., Menlo Park, CA (US)

(72) Inventors: Brett N. ANDERSON, Menlo Park, CA (US); Martin HUBER, Menlo Park, CA (US); Daniel KOSLOVER, Menlo Park, CA (US); Jan F. SIMONS, Menlo Park, CA (US)

(73) Assignee: Quantapore, Inc., Menlo Park, CA (US)

(21) Appl. No.: 15/566,171

(22) PCT Filed: Jun. 6, 2016
(86) PCT No.: PCT/US16/36048

§ 371 Date: Oct. 12, 2017

Related U.S. Application Data
(60) Provisional application No. 62/173,350, filed on Jun. 9, 2015.

(51) Int. Cl. G01N 21/64; C12Q 1/6869; G01N 33/542

(52) U.S. Cl. CPC G01N 21/6428; C12Q 1/6869; G01N 2021/6441; G01N 2021/6432; G01N 33/542

(57) ABSTRACT

The invention is directed to devices and methods for optically based nanopore analysis which employ FRET signaling wherein at least one member of a FRET pair is mobile within a lipid bilayer containing one or more nanopores. In some embodiments, mobile FRET donors are constrained to a lipid bilayer so that they may continuously diffuse into and within a FRET distance of acceptor-labeled analytes entering or exiting a nanopore so that bleached or degraded FRET donors are continuously replaced.

### CROSS-REFERENCE TO RELATED APPLICATION

The present application claims priority to U.S. Provisional Application No. 62/173,350, filed 9 Jun. 2015, which is incorporated by reference herein in its entirety.

### BACKGROUND

DNA sequencing technologies developed over the last decade have revolutionized the biological sciences, e.g. Lerner et al, The Auk, 127: 4-15 (2010); Metzker, Nature Review Genetics, 11: 31-46 (2010); Holt et al, Genome Research, 18: 839-846 (2008); and have the potential to revolutionize many aspects of medical practice in coming years, e.g. Voelkerding et al, Clinical Chemistry, 55: 641-658 (2009); Anderson et al, Genes, 1: 38-69 (2010); Freeman et al, Genome Research, 19: 1817-1824 (2009); Tucker et al, Am. J. Human Genet., 85: 142-154 (2009). However, to realize such potential there are still a host of challenges that must be addressed, including reduction of per-run sequencing cost, simplification of sample preparation, reduction of run time, increasing sequence read lengths, improving data analysis, and the like, e.g. Baker, Nature Methods, 7: 495-498 (2010); Kircher et al, Bioessays, 32: 524-536 (2010); Turner et al, Annual Review of Genomics and Human Genetics, 10: 263-284 (2009). Single molecule sequencing using nanopores may address some of these challenges, e.g., Maitra et al, Electrophoresis, 33: 3418-3428 (2012); Venkatesan et al, Nature Nanotechnology, 6: 615-624 (2011); however, this approach has its own set of technical difficulties, such as, reliable nanopore fabrication, control of DNA translocation rates, nucleotide discrimination, detection of electrical signals from large arrays of nanopore sensors, and the like, e.g. Branton et al, Nature Biotechnology, 26(10): 1146-1153 (2008); Venkatesan et al (cited above).

Optical detection of nucleotides has been proposed as a potential solution to some of the technical difficulties in the field of nanopore sequencing, e.g. Huber, U.S. Pat. No. 8,771,491; Russell, U.S. Pat. No. 6,528,258; Pittaro, U.S. patent publication 2005/0095599; Joyce, U.S. patent publication 2006/0019259; Chan, U.S. Pat. No. 6,355,420; McNally et al, Nano Lett., 10(6): 2237-2244 (2010); and the like. Several promising approaches to optical detection exploit fluorescence resonance energy transfer (FRET) for generation of localized analyte-specific optical signals at a nanopore, which can be distinguished from signals at other nanopores. Unfortunately, some of the best candidates for FRET donors require difficult design trade-offs. In particular, quantum dots as donors have favorable stability, but have size and structural properties that make them difficult to position on nanopore arrays. Organic FRET donors have convenient sizes and chemistries available for direct attachments to, for example, protein nanopores, but they lack chemical stability for prolonged use and soon become “bleached” and inoperable.

In view of the above, it would be advantageous to nanopore sensor technology in general and its particular applications, such as optically based nanopore sequencing and analyte detection, if methods and devices were available for addressing the limitations imposed by FRET donors.

### SUMMARY OF THE INVENTION

The present invention is directed to devices and methods for efficient optical detection and analysis of analytes, such as polymers (including polynucleotides) using nanopore arrays.

In one aspect, the invention is directed to devices for detecting an analyte comprising the following elements: (a) a solid phase membrane separating a first chamber and a second chamber, the solid phase membrane having at least one aperture connecting the first chamber and the second chamber, (b) a lipid bilayer disposed on at least one surface of the solid phase membrane, the lipid bilayer comprising a concentration of donors of a fluorescence resonance energy transfer (FRET) pair, the donors being mobile within the lipid bilayer; and (c) a nanopore immobilized in the aperture, the nanopore having a bore with an entrance and an exit and the nanopore interacting with the lipid layer to form a seal with the solid phase membrane in the aperture so that fluid communication between the first chamber and the second chamber occurs solely through the bore of the nanopore; wherein the concentration of donors in the lipid bilayer is selected so that whenever an analyte labeled with an acceptor of the FRET pair exits or enters the bore of the nanopore with a predetermined likelihood at least one donor is within a FRET distance of the acceptor.

In some embodiments, the nanopore is a protein nanopore. In some embodiments, concentration of donors is selected so that an expected frequency of donors coming within a FRET distance of an entrance or an exit of said bore (depending on which surface of the solid phase membrane the lipid bilayer is disposed) is equal to or greater than a frequency with which the acceptors enter or exit said bore, respectively. In other embodiments, an expected frequency of donors coming within a FRET distance of an entrance or exit of said bore is equal to or greater than ten times the frequency with which acceptors enter or exit said bore, respectively.

In another aspect the invention is directed to methods of determining a nucleotide sequence of a polynucleotide comprising the steps of: (a) providing a device comprising: (i) a solid phase membrane separating a first chamber and a second chamber, the solid phase membrane having at least one aperture connecting the first chamber and the second chamber, and the solid phase membrane having a lipid bilayer on at least one surface; (ii) a protein nanopore immobilized in the aperture, the protein nanopore having a bore, the protein nanopore contacting and extending through the lipid bilayer so that the first chamber and the second chamber are in fluid communication through the bore; and (iii) donors of one or more FRET pairs diffusably disposed in the lipid bilayer; (b) translocating the polynucleotide through the protein nanopore so that acceptor labels attached to the polynucleotide pass sequentially therethrough and so that whenever an acceptor label exits the bore of the protein nanopore at least one donor with a predetermined likelihood is within a FRET distance of such acceptor label; and (c) determining a nucleotide sequence of the polynucleotide by a sequence of FRET interactions between donors and acceptor labels exiting the bore or the protein nanopore. In some embodiments, donors are capable of FRET only within a predetermined proximity of an exit of a bore of a nanopore. In some embodiments, donors are ion-sensitive dyes that are capable of FRET only when a predetermined ion is present at a predetermined concentration.

The present invention is exemplified in a number of implementations and applications, some of which are summarized below and throughout the specification.

### BRIEF DESCRIPTION OF THE DRAWINGS

FIGS. 1A-1B illustrates a design of a hybrid nanopore configured for FRET detection using a donor attached directly to the stem of a protein nanopore.

FIGS. 1C-1F illustrate various embodiments of a hybrid nanopore employing mobile FRET donors in a lipid bilayer.

FIGS. 2A, 2B and 2C illustrate embodiment where one or more nanopores, e.g. protein nanopores, may occupy a single aperture of a solid phase membrane.

FIGS. 3A-3B further illustrate embodiments of a hybrid nanopore employing mobile FRET donors in a lipid bilayer wherein the mobile donors are ion-sensitive dyes (calcium ion shown) that are capable of FRET only in the presence of a predetermined concentration of predetermined ion.

FIG. 3C illustrates an embodiment wherein donors are lipophilic and are delivered to a lipid bilayer adjacent to a nanopore by a carrier molecule. After illumination FRET donors, which may be bleached, diffuse away from the nanopore in the lipid bilayer.

FIG. 4A illustrates the operation of a device of the invention.

FIG. 4B illustrates a surface of a solid phase membrane on which a lipid bilayer containing a single type of mobile donor (122) is disposed.

FIG. 4C illustrates a surface of a solid phase membrane on which a lipid bilayer containing two types of mobile donors (122 and 123) is disposed.

### DETAILED DESCRIPTION OF THE INVENTION

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention. For example, particular nanopore types and numbers, particular labels, FRET pairs, detection schemes, fabrication approaches of the invention are shown for purposes of illustration. It should be appreciated, however, that the disclosure is not intended to be limiting in this respect, as other types of nanopores, arrays of nanopores, and other fabrication technologies may be utilized to implement various aspects of the systems discussed herein. Guidance for aspects of the invention is found in many available references and treatises well known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2^{nd} edition (Wiley Interscience, 1995); Bard and Faulkner, Electrochemical Methods: Fundamentals and Applications, 2^{nd} edition (Wiley, 2000); Lakowicz, Principles of Fluorescence Spectroscopy, 3^{rd} edition (Springer, 2006); Hermanson, Bioconjugate Techniques, Second Edition (Academic Press, 2008); and the like, which relevant parts are hereby incorporated by reference.

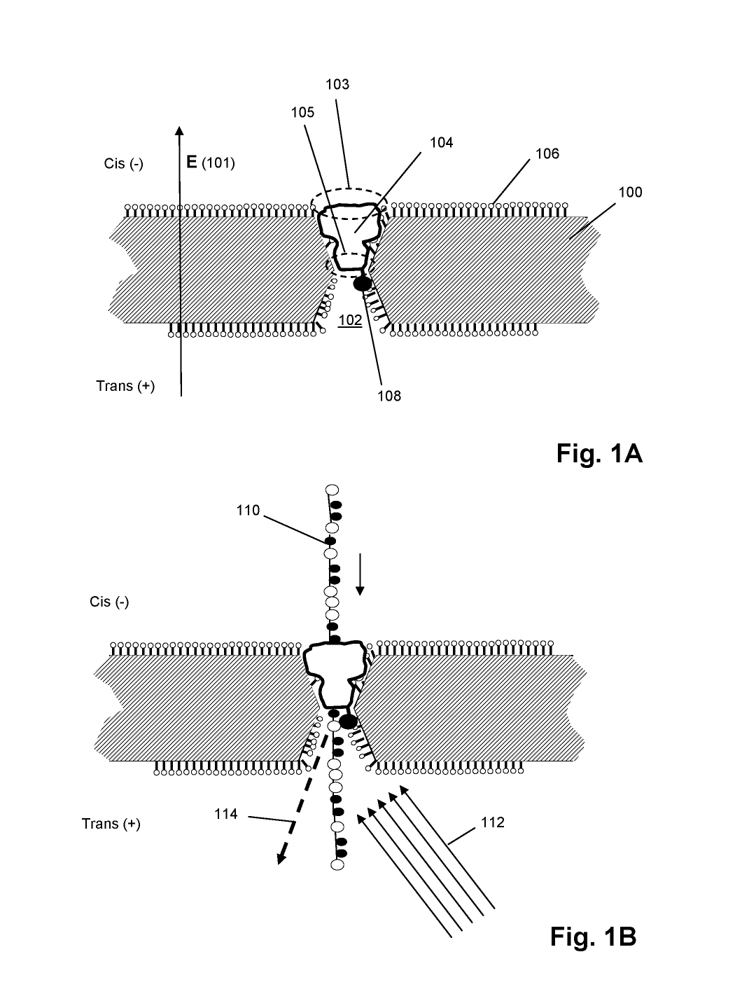

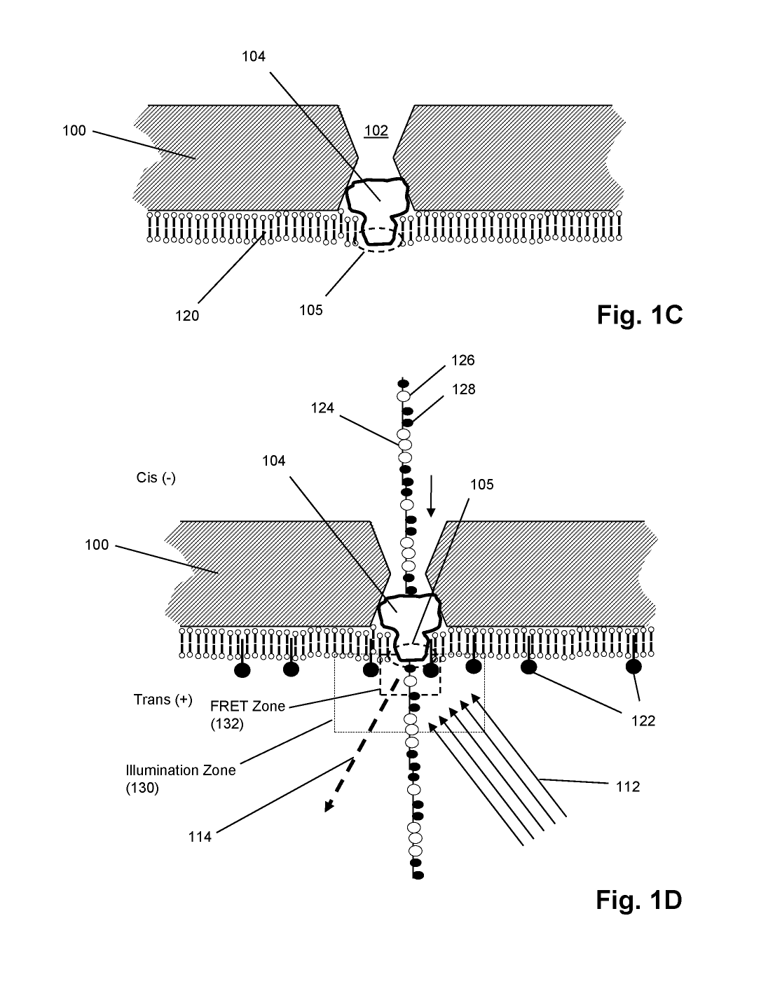

In part the invention addresses the problem of donors becoming bleached or degraded upon extended illumination in the field of FRET-based detection of labeled analytes using nanopores. In one aspect, multiple donors are made available for FRET interactions at each nanopore by disposing nanopores in a lipid bilayer containing donors that are constrained to the lipid bilayer but are otherwise capable of diffusing freely within the lipid bilayer. Thus, each nanopore (which is in a fixed position) is replenished continuously with donors as new donors diffuse to within a FRET distance of the nanopore. As illustrated in FIGS. 1C-1F, a lipid bilayer may be disposed on a cis(−) side or a trans(+) of a solid phase membrane containing nanopores. In some embodiments hybrid nanopores are employed that comprise solid phase membranes with one or more orifices in which protein nanopores are disposed. In such embodiments, in addition to lipid bilayers disposed on either cis(−) or trans(+) sides (or surfaces) of solid phase membranes, protein nanopores may have different orientations within a lipid bilayer; namely, for protein nanopores having stem and cap structures along the axis of a nanopore's bore, or lumen, the cap may be oriented toward the solid phase membrane, as illustrated in FIG. 1C, or the cap may be oriented away from the solid phase membrane, as illustrated in FIG. 1E.

In atypical configuration, illustrated in FIGS. 1A-1B, protein nanopore (104) may be immobilized in aperture, or orifice, (102) on the cis(−) side of solid phase membrane (100), which may include coating (106), e.g. a lipophilic or hydrophobic coating, to facilitate placement of protein nanopore (104). The cis(−) side is defined in this configuration as the side of solid phase membrane (100) from which nucleic acid analytes enter a nanopore, for example, under the influence of an electrical field (although this is not meant to limit devices and methods of the invention to the use of electric fields to translocate analytes through nanopores). The trans(+) side is defined in this configuration as the side of solid phase membrane (100) to which nucleic acid analytes exit a nanopore. In typical embodiments, single stranded nucleic acid analytes are disposed on the cis(−) side of solid phase membrane (100) in an electrolyte solution under conditions, e.g. pH, that give the single stranded nucleic acids a net negative charge, so that upon the application of electric field, E (101), the single stranded nucleic acid analytes are driven through nanopores in solid phase membrane (100).

Illustrated in the FIGS. 1A and 1B is an exemplary protein nanopore, such as hemolysin, which has a cap end (103) and stem end (105), for example, as described in Song et al, Science, 274: 1859-1866 (1996). In this configuration, because of steric constraints of orifice (102), donor (108) attached to stem end (105) of protein nanopore (104) is limited to an organic dye, or a similarly sized molecule that is not sterically excluded from orifice (102). Upon translocation of nucleic acid analyte (110) through protein nanopore (104), acceptor-labeled monomers of analyte (110) move within a FRET distance of donor (108), which excites adjacent acceptors and generates signal (114) whenever donor (108) is illuminated by excitation beam (112). If donor (108) is a conventional organic dye, it may be rapidly degraded or bleached by continuous or repeated excitation, thereby limiting the application of the illustrated configuration for analyzing long and/or multiple nucleic acid strands.

In some embodiments, the invention addresses this problem by disposing nanopore (104), for example, a protein nanopore, into orifice (102) on the trans(+) side of solid phase membrane (100) with its stem end (105) embedded in lipid bilayer (120) disposed on the trans(+) surface of solid phase membrane (100), as illustrated in FIG. 1C. Lipid bilayer (120) provides a medium for containing donors (122) that are lipophilic or that contain a lipophilic moiety that constrains them spatially to lipid bilayer (120), but otherwise allows them to move within lipid bilayer (120). Donors (122) may be detergent-like, that is, having a lipophilic portion and a hydrophilic portion so that donors (122) are partially immersed in lipid bilayer (120)(as illustrated in FIG. 1D), or donors (122) may be completely or predominantly lipophilic so that they are fully immersed in lipid bilayer (120)(not shown). Donors may be hydrophilic and directly attached to the hydrophilic part of a lipid bilayer, but allows them to move within the lipid bilayer through the lipid component they are attached to. In some embodiments, donors (122) move randomly by diffusion within lipid bilayer (120). The rate of such movement may be adjusted by adding components, such as lipophilic polymers, or other compounds, such as cholesterol, to lipid bilayer (120) that inhibit movement of donors (122) or by providing donors (122) with higher or lower molecular weights to reduce the rate of movement or to increase the rate of movement, as desired, respectively. In some embodiments, more than one kind of donor (122) may be provided. For example, donors (122) may include donors that have different absorption bands or that have different emission bands. Such differing donors may provide a wider selection of FRET pairs for use in the invention. In some embodiments, donors (122) include a plurality of different kinds of donor, each having a different and substantially non-overlapping emission band. In accordance with the invention, donors are excited only within an illumination zone. Because donors are mobile within lipid bilayer (120), donors that degrade or are bleached diffuse away from end (105) of nanopore (104) and are replenished by “fresh” donors (i.e. capable of participating in a FRET interaction with an acceptor) that diffuse into the illumination region. An illumination zone may be an area encompassing one or more nanopores or it may be generated by intermittent illumination. That is, in the latter case, an illumination zone may encompass the entire surface of solid phase membrane (100) and nanopores contained therein, but an excitation beam is directed to it periodically so that there are alternating intervals of excitation and non-excitation (or darkness). In some embodiments, degraded or bleached donors are replenished essentially continuously by selecting a concentration of donors in lipid bilayer (120) sufficiently high so that new donors enter the FRET zone at a frequency equivalent to or greater than the frequency by which new acceptors enter the FRET zone, for example, at a frequency 5 times greater, or 10 times greater, or 100 times greater than the frequency by which new acceptors enter the FRET zone. In alternative embodiments, degraded or bleached donors are replenished within a FRET zone by providing “dark” intervals when no excitation beam is present and during which new donors may diffuse into the FRET zone; that is, degraded and bleached donors are replenished by intermittently illuminating illumination zone (130) with an excitation beam, so that during non-illuminated periods donors are replenished by diffusion.

As illustrated in FIGS. 1E and IF, in some embodiments, the degradation and bleaching problem may be addressed by disposing nanopore (104), for example, a protein nanopore, into orifice or aperture (102) on the cis(−) side of solid phase membrane (100) with its stem end (105) embedded in lipid bilayer (120) disposed on the cis(−) surface of solid phase membrane (100). As above, mobile donors constrained to lipid bilayer (120) continually replenish donors in a FRET zone by diffusion. Operation of such embodiments is similar to those of FIGS. 1C and 1D. Typically, solid phase membrane (100) is transparent or nearly transparent so that similar, if not identical, illumination and detection schemes may be used in both embodiments.

One of ordinary skill in the art would recognize and appreciate that while the above examples illustrate the invention with a protein nanopore, other embodiments may include non-protein nanopores for aligning acceptor-labeled polymer analytes for excitation with mobile donors in a lipid bilayer.

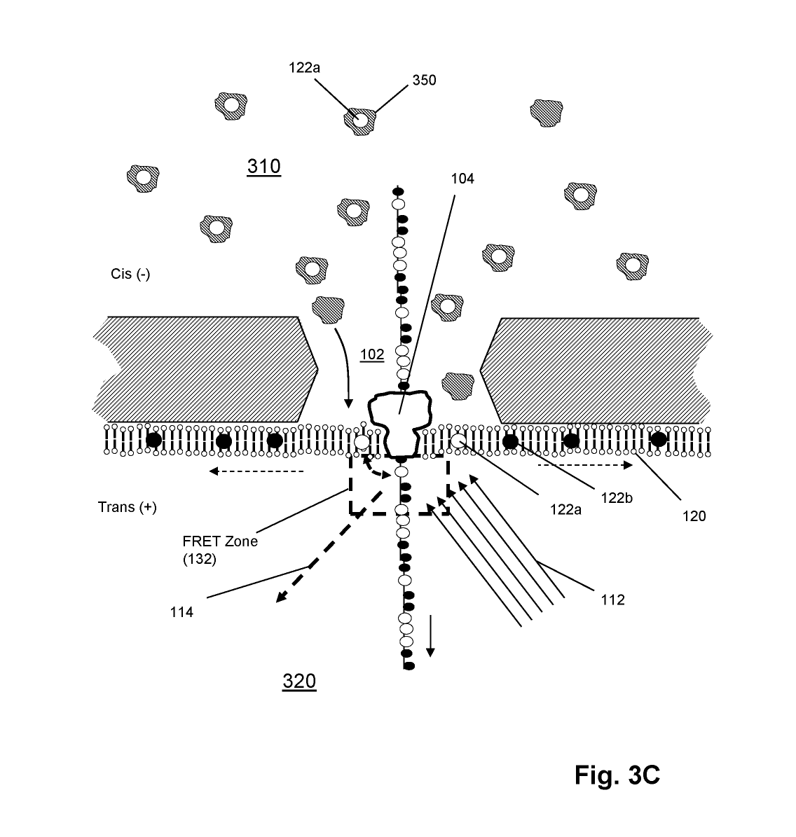

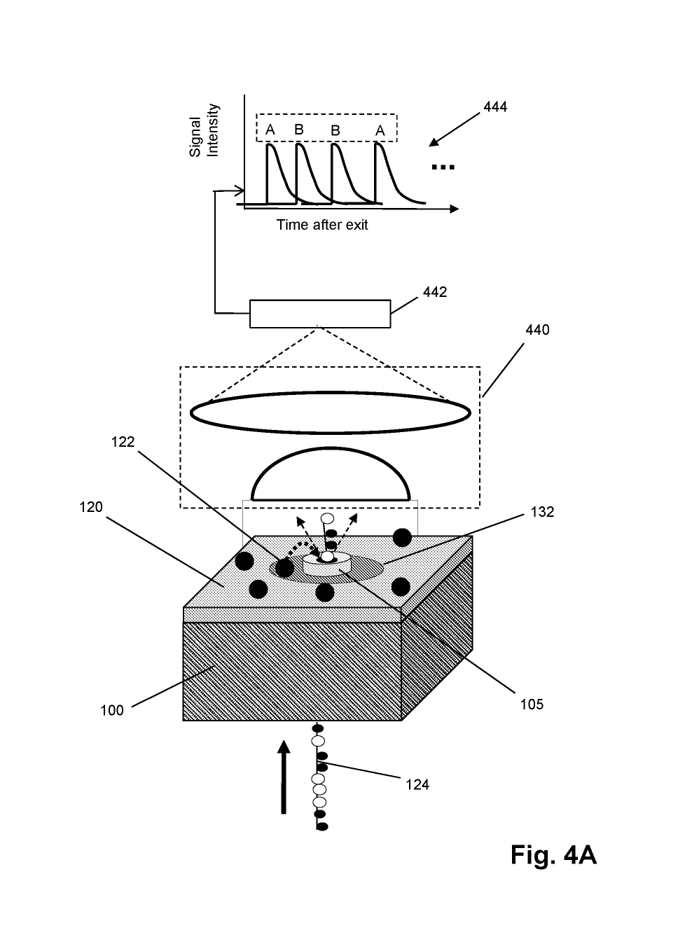

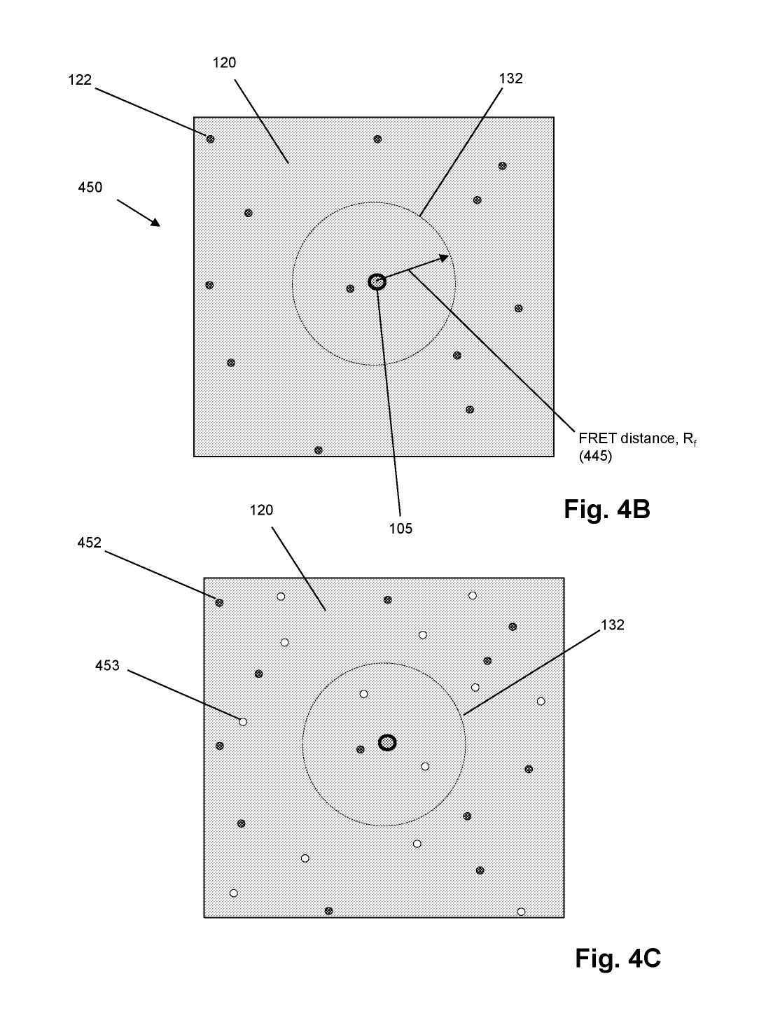

The operation of the above embodiments of the invention is illustrated in FIGS. 1D and 4A. FIG. 1D illustrates polymer analyte (124) whose monomers are labeled with two different acceptor molecules (126, white; and 128, black) translocating through protein nanopore (104) disposed in solid phase membrane (100) in accordance with the invention. Excitation beam (112) illuminates a region around stem end (105) of protein nanopore (104) (referred to herein as an illumination zone, shown in FIG. 1D as (130)). Adjacent to stem end (105) there is a FRET zone (132) within which a FRET interaction takes place between a donor and an acceptor whenever a donor (122) is in FRET zone (132) and an acceptor label of polymer analyte (124) is in FRET zone (132). As a result of such FRET interaction, FRET signal (114) is produced, which is detected.

These concepts are further illustrated in FIG. 4A. Excitation of donors (122) may be accomplished using a total internal reflectance fluorescence (TIRF) microscope system. Such a system may also serve as a detection system, as illustrated diagrammatically by (440). Labels on polymer analyte (124) may be excited directly or indirectly using FRET donor-acceptor pairs. Detector (442) collects optical signals and convert them into values that can be displayed, such as the curves (444). An objective of the analytical systems such as illustrated in FIG. 4A is to use the optical signals generated by the optical labels to identify a sequence of monomers of polymer analyte (124). For example, in the single nanopore case, base (or monomer) calls can be made successfully in a 2-label system; that is, A, B (i.e. “not-A”), B and A (444).

In some embodiments, a concentration of donors (122), diffusion coefficient, and operating conditions are selected so that the expected frequency of donors entering a FRET zone is equal to or greater than the frequency of acceptors entering the FRET zone. Such concentration will depend on the mobility of donors (122), the rate of translocation of polymer analyte (124), the spacing of acceptor labels on polymer analyte (124), the nature of the distribution of donors (122) in lipid bilayer (120), and like factors well-known to those of ordinary skill in the art. In some embodiments, donors will have an expected occupancy time in a FRET zone which depends on the above-listed factors. In some embodiments, the above-listed factors are selected so that donor occupancy time is greater than acceptor expected occupancy time in the FRET zone due to translocation speed. In other embodiments, the above-listed factors are selected so that donor occupancy time is at least three times greater than acceptor expected occupancy time; in other embodiments; the above-listed factors are selected so that donor occupancy time is at least ten times greater than acceptor expected occupancy time; in other embodiments; the above-listed factors are selected so that donor occupancy time is at least twenty times greater than acceptor expected occupancy time. In other embodiments, the above-listed factors are selected so that donor occupancy time is less than the expected bleaching time of the donor.

FIG. 4B provides an additional view of a surface of a solid phase membrane (450) with lipid bilayer (120) and mobile donors (122) immersed in, or constrained thereto. For example, lipid bilayer (120) may be on a trans surface of membrane (450) when analytes are negatively charged polynucleotides. Also shown is a FRET zone (132) defined by radius (445) in a proximity of nanopore exit (105). FIG. 4C illustrates a similar view of an embodiment wherein more than one mobile donors (452 and 453) are constrained to, and mobile in, lipid bilayer (120). In some embodiments, a plurality of different kinds of mobile donors may be used that correspond to different FRET pairs, so that different analytes may be detected by interactions between their own FRET pairs. In other embodiments, each mobile donor may be capable of a FRET interaction with one or more acceptors on one or more analytes. For example, an embodiment may comprise two mobile donors, say, X and Y, where in a device for determining nucleotide sequences, donor X may have a FRET interaction with acceptors on nucleotides A and G, and donor Y may have a FRET interaction with acceptors on nucleotides C and T. In some embodiments, there may be a plurality of mobile donors; in other embodiments, such plurality may be in the range of from 2 to 5 mobile donors; in still other embodiments, such plurality may be in the range of from 2 to 3 mobile donors.

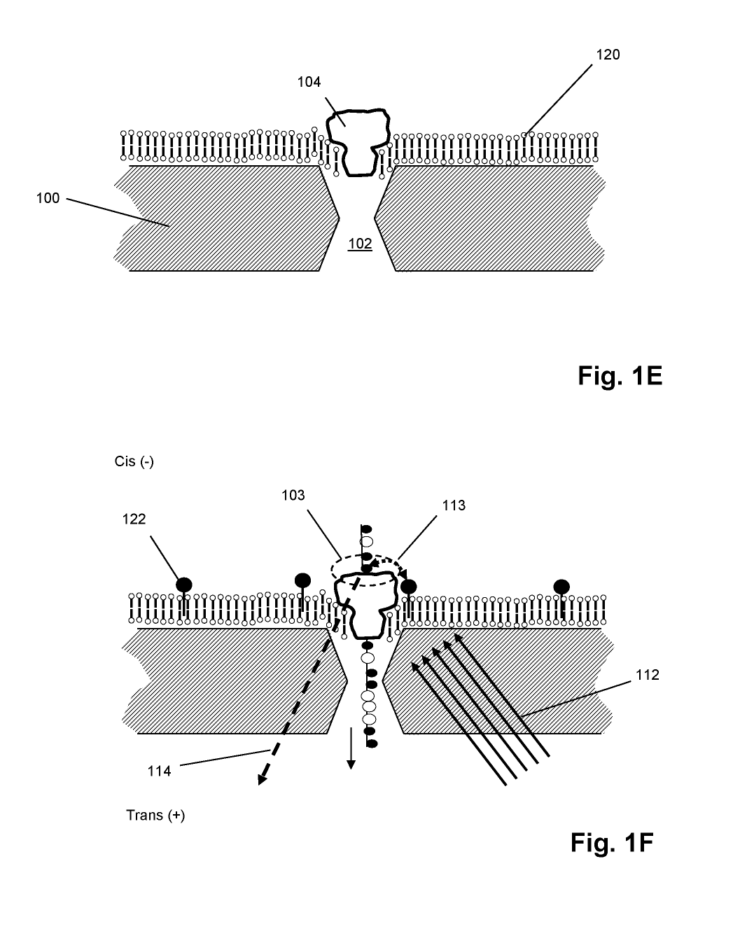

In some embodiments, as noted further below, cross-sectional dimensions (200) of apertures, such as (102) in FIGS. 2A-2C may be considerably larger than that of a protein nanopore, such as illustrated by (104) in FIG. 2A. In such embodiments, protein nanopore (104) may diffuse freely with the boundaries (202) of the cross section of aperture (102). With such larger aperture cross sectional areas more than one protein nanopore may be disposed in a single aperture, as illustrated in FIGS. 2B and 2C by (104a and 104b). In some embodiments, a plurality of protein nanopores in a single aperture is advantageous, since the duty cycles of apertures may be effectively increased since there would be reduced wait times for the next analyte to be processed by an aperture. The concentration of protein nanopores and the concentration of analytes may be selected to optimize processing throughput, especially, for example, when analytes are polynucleotides.

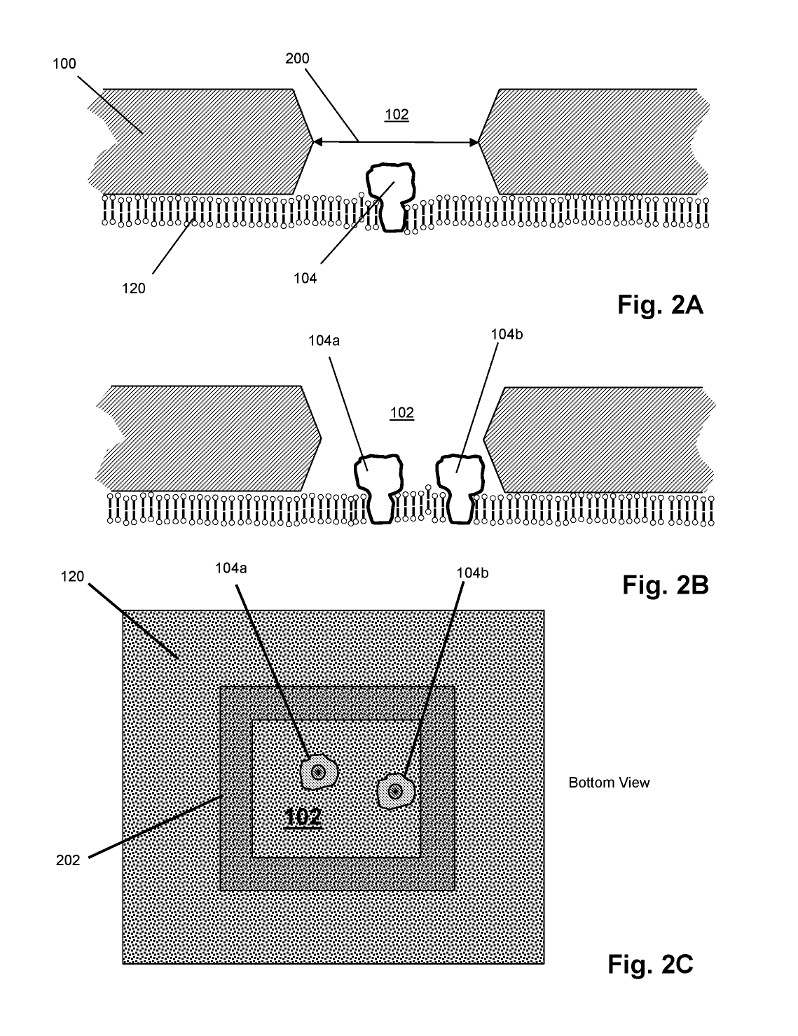

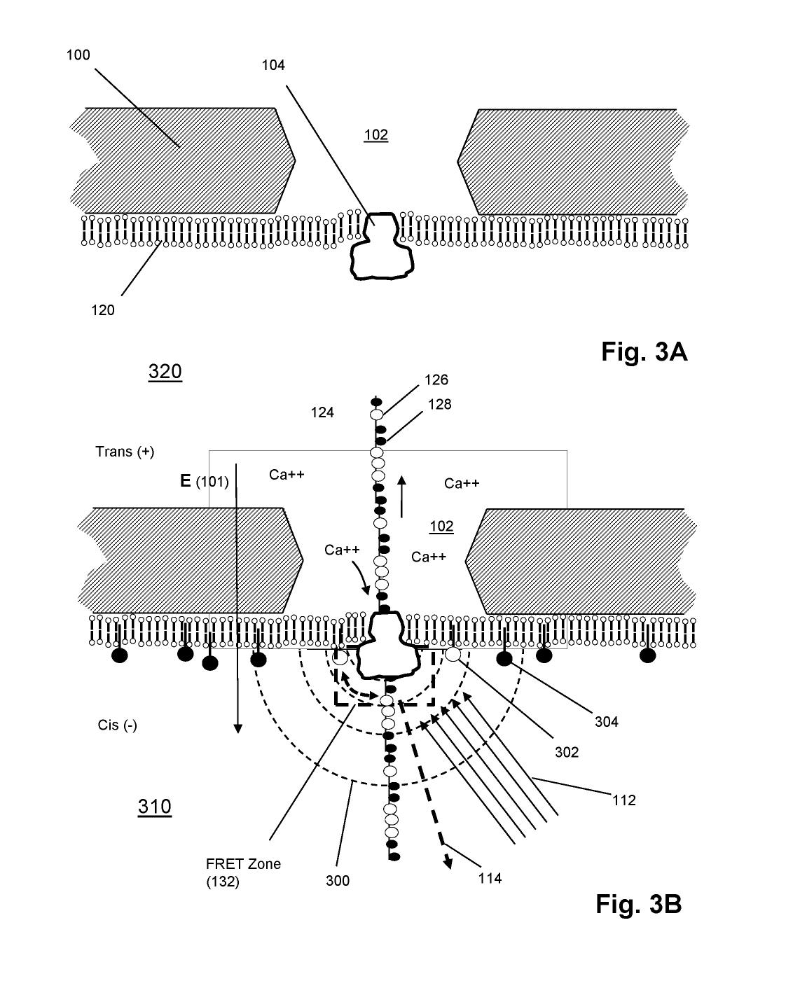

In some embodiments, as exemplified in FIGS. 3A-3C, donors are employed that are capable of FRET only within a predetermined proximity of a nanopore. In some embodiments, a predetermined proximity may be the same or greater than a dimension of a FRET zone; in other embodiments, a predetermined proximity may be less than the distance or half the distance to an adjacent nanopore. In some embodiments, a predetermined proximity is a radial distance from an exit or entrance of a bore of a nanopore which is less than 1 μm; or which is less than 500 nm. Such embodiments are advantageous in that background fluorescence from donors outside the proximity of apertures is reduced or eliminated. One such embodiment is illustrated for a protein nanopore (104 in FIG. 3A) in lipid bilayer (120) spanning an aperture (102) in solid phase membrane (100). In this embodiment, ion-sensitive donors (302-FRET-capable shown in white, and 304-FRET-inert, shown in black) are employed that are capable of FRET only under conditions of an ion concentration that exceeds a predetermined value. Locally elevated concentrations of a desired ion, such as calcium, Ca⁺⁺, may be created at an entrance or exit of a nanopore with initial conditions where a first chamber (310) (on one side of solid phase membrane (100)) has a concentration of ions below the predetermined value, for example, zero or substantially zero concentration, and a second chamber (320) has a concentration of ions above the predetermined value. Guidance in establishing and measuring such gradients is found in Anderson et al, ACS Nano, 8(11): 11836-11845 (2014), which is incorporated herein by reference. In some embodiments, first chamber (310) and second chamber (320) may correspond to a cis and trans orientation of electrical field (101), respectively. Average or bulk ion concentrations of the chambers may be maintained by using chelator compounds, e.g. EDTA for Ca⁺⁺. However, diffusion of ions from chamber two to chamber one will occur through nanopores, e.g. (104), or by the addition of ion-selective ionophores to aperture (102), so that locally elevated concentrations of ions (300) are created in the proximities of the exits of nanopores (104). A predetermined concentration of ions in chamber two is selected so that an elevated local concentration (300) renders lipid-bound ion-sensitive donors (304) capable of FRET whenever such donor is within a FRET distance (132) of a nanopore exit. Exemplary ion-sensitive donors include calcium ion-sensitive Fluor-4 or related dyes derivatized with a lipophilic anchor moiety using conventional techniques. One of ordinary skill in the art would understand that a gradient of any chemical activator of a fluorescent donor may be used in accordance with the above embodiments of the invention. Other chemical gradients include, but are not limited to, pH, electron accepting compounds or oxidizing agents, electron donating compounds or reducing agents, or developers or activators of fluorescent leuco dyes, and the like.

FIG. 3C illustrates another embodiment in which donor fluorescence is minimized outside a proximity of apertures. Lipid-sensitive and FRET-capable dyes (122a) are delivered from first chamber (310) by carrier (350) to lipid bilayer (120) in the proximity of nanopore (104), for example, defined in this example by the cross-sectional area of aperture (102). In the proximity of nanopore (104), dyes (122a) are illuminated by excitation beam (112) and undergo FRET with an acceptor within FRET distance (132), which generates fluorescent signal (114). Dye (122a), which may be bleached and inert after a FRET event (122b) diffuses out of proximity of aperture (102). Exemplary carriers (350) include cyclodextrin and like molecules.

The FRET distance (and therefore “FRET zone”) around a nanopore, may be defined operationally such as the distance from a nanopore within which a FRET interaction occurs with ninety-nine percent probability, or other selected value, for example, ninety percent, or ninety-five percent.

### Nanopores and Nanopore Arrays

Nanopores used with the invention may comprise solid-state nanopores, protein nanopores, or hybrid nanopores comprising protein nanopores or organic nanotubes such as carbon nanotubes, configured in a solid-state membrane, or like framework. In some embodiments, functions and properties of nanopores include (i) constraining analytes, particularly polymer analytes, to pass through a detection zone in sequence, or in other words, so that monomers pass a detection zone one at a time, or in single file, (ii) compatibility with a translocating means (if one is used), that is, whatever method is used to drive an analyte through a nanopore, such as an electric field, and optionally in some embodiments, (iii) suppression of fluorescent signals within the lumen, or bore, of the nanopore. In some embodiments, nanopores are used in connection with the methods and devices of the invention in the form of arrays, either regular arrays, such as rectilinear arrays of a plurality nanopores in a planar support or membrane, or random arrays, for example, where a plurality of nanopores are spaced in accordance with a Poisson distribution in a planar support or membrane, or an array of clusters of nanopores, which may be disposed regularly on a planar surface.

Solid phase membranes from which nanopores and/or nanopore arrays are constructed may be fabricated in a variety of materials including but not limited to, silicon nitride (Si₃N₄), silicon dioxide (SiO₂), Hafnium oxide (HfO₂), Titanium oxide (TiO₂), Aluminum oxide (Al₂O₃) or combinations thereof and the like. The fabrication of nanopores and/or nanopore arrays and their operation (e.g. sample handling systems, detection systems, and the like) for analytical applications, such as DNA sequencing, are disclosed in the following exemplary references which are incorporated by reference: Russell, U.S. Pat. No. 6,528,258; Feier, U.S. Pat. No. 4,161,690; Ling, U.S. Pat. No. 7,678,562; Hu et al, U.S. Pat. No. 7,397,232; Golovchenko et al, U.S. Pat. No. 6,464,842; Chu et al, U.S. Pat. No. 5,798,042; Sauer et al, U.S. Pat. No. 7,001,792; Su et al, U.S. Pat. No. 7,744,816; Church et al, U.S. Pat. No. 5,795,782; Bayley et al, U.S. Pat. No. 6,426,231; Akeson et al, U.S. Pat. No. 7,189,503; Bayley et al, U.S. Pat. No. 6,916,665; Akeson et al, U.S. Pat. No. 6,267,872; Meller et al, U.S. patent publication 2009/0029477; Howorka et al, International patent publication WO2009/007743; Brown et al, International patent publication WO2011/067559; Meller et al, International patent publication WO2009/020682; Polonsky et al, International patent publication WO2008/092760; Van der Zaag et al, International patent publication WO2010/007537; Yan et al, Nano Letters, 5(6): 1129-1134 (2005); Iqbal et al, Nature Nanotechnology, 2: 243-248 (2007); Wanunu et al, Nano Letters, 7(6): 1580-1585 (2007); Dekker, Nature Nanotechnology, 2: 209-215 (2007); Storm et al, Nature Materials, 2: 537-540 (2003); Wu et al, Electrophoresis, 29(13): 2754-2759 (2008); Nakane et al, Electrophoresis, 23: 2592-2601 (2002); Zhe et al, J. Micromech. Microeng., 17: 304-313 (2007); Henriquez et al, The Analyst, 129: 478-482 (2004); Jagtiani et al, J. Micromech. Microeng., 16: 1530-1539 (2006); Nakane et al, J. Phys. Condens. Matter, 15 R1365-R1393 (2003); DeBlois et al, Rev. Sci. Instruments, 41(7): 909-916 (1970); Clarke et al, Nature Nanotechnology, 4(4): 265-270 (2009); Bayley et al, U.S. patent publication 2003/0215881; and the like.

Briefly, in one aspect, a 1-50 nm channel is formed through a substrate, usually a membrane, through which an analyte, such as DNA, is induced to translocate. The solid-state approach of generating nanopores offers robustness and durability as well as the ability to tune the size and shape of the nanopore, the ability to fabricate high-density arrays of nanopores on a wafer scale, superior mechanical, chemical and thermal characteristics compared with systems solely employing lipid bilayer supports, and the possibility of integrating with electronic or optical readout techniques. Biological nanopores on the other hand provide reproducible narrow bores, or lumens, especially in the 1-10 nanometer range, as well as techniques for tailoring the physical and/or chemical properties of the nanopore and for directly or indirectly attaching groups or elements, such as lipophilic anchoring groups, fluorescent labels, which may be FRET donors or acceptors, or the like, by conventional protein engineering methods. Protein nanopores typically rely on delicate lipid bilayers for mechanical support, and the fabrication of solid-state nanopores with precise dimensions remains challenging. Combining solid-state nanopores with a biological nanopore overcomes some of these shortcomings, especially the precision of a biological pore protein with the stability of a solid state nanopore. For optical read out techniques lipid hybrid nanopores provide a precise location of the nanopores by deploying regularly spaced apertures which simplifies the data acquisition greatly by constraining the lateral diffusion of the protein nanopores to the cross sections of the apertures (as illustrated, for example, in FIG. 2C).

In some embodiments, a hydrophilic coating is optional in that the surface of the solid phase membrane is sufficiently hydrophilic itself so that a lipid bilayer adheres to it stably. The at least one aperture will have an inner surface, or wall, connected to, or contiguous with the surfaces of the solid phase membrane. In some embodiments, the at least one aperture will be a plurality of apertures, and the plurality of apertures may be arranged as a regular array, such as a rectilinear array of apertures, the spacing of which depending in part on the number and kind of FRET pairs employed and the optical detection system used. In particular, in some embodiments, a plurality of apertures is arranged in an array of clusters of apertures. Each of the apertures has a diameter, which in some embodiments is such that a protein nanopore is substantially immobilized therein. In some embodiments, substantially immobilized means that a protein nanopore may move no more than 5 nm in the plane of the solid phase membrane relative to the wall of the aperture. In another embodiment, substantially immobilized means that a protein nanopore may move no more than 35 nm in the plane of the solid phase membrane relative to the wall of the aperture. In yet another embodiment, substantially immobilized means that a protein nanopore may move no more than 55 nm in the plane of the solid phase membrane relative to the wall of the aperture. In yet another embodiment, substantially immobilized means that a protein nanopore may move no more than 100 nm in the plane of the solid phase membrane relative to the wall of the aperture. In yet another embodiment, substantially immobilized means that a protein nanopore may move no more than 200 nm in the plane of the solid phase membrane relative to the wall of the aperture. The protein nanopores each have a bore, or passage, or lumen, which permits fluid communication between the first and second chambers when the protein nanopore is immobilized in an aperture. Generally, the bore is coaxially aligned with the aperture. One function of the hydrophilic layer is to provide a surface to retain a lipid bilayer which can span one or a multitude of apertures. The hydrophilic layer can be on one or on both sides of the solid phase membrane. Such lipid bilayers, in turn, permit disposition and immobilization of a protein nanopore within an aperture in a functional conformation and in a manner that forms a seal with the wall of the aperture. In some embodiments, such seal also prevents electrical current passing between the first and second chambers around the protein nanopore. In some embodiments, charged analytes are disposed in an electrolyte solution in the first chamber and are translocated through the bore(s) of the protein nanopore(s) into an electrolytic solution in the second chamber by establishing an electrical field across the solid phase membrane. For convenience of manufacture, in some embodiments the hydrophilic coating will be on one or both surfaces of the solid phase membrane and the wall(s) of the aperture(s). Hydrophilic surfaces can be generated by chemically modifying the solid support membrane by direct deposition of a layer of hydrophilic reagents. Alternatively, the solid surface can be treated by a plasma which renders the surface hydrophilic. In another embodiment a harsh acidic or basic treatment can render a surface such as SiN or SiO2 hydrophilic. By incorporating certain groups in the hydrophilic layer that specifically bind to the lipids in the bilayer a so called tethered lipid bilayer with increased stability can be generated. All of the above techniques are within the scope of this invention.

In some embodiments, the solid phase membrane may be treated with a low energy ion beam to bleach its autofluorescence, e.g. as described in Huber et al, U.S. patent publication 2013/0203050, which is incorporated herein by reference.

In some embodiments, the present invention employs a hybrid nanopore, particularly for optical-based nanopore sequencing of polynucleotides. Such embodiments comprise a solid-state orifice, or aperture, into which a protein biosensor, such as a protein nanopore or any other transmembrane protein is stably inserted.

Once stable hybrid nanopores have formed, single stranded, fluorescently labeled (or acceptor labeled) DNA is added to the cis chamber. The applied electric field forces the negatively charged ssDNA to translocate through the hybrid nanopore during which the labeled nucleotides get in close vicinity of a donor fluorophore immersed in the lipid bilayer.

Solid state, or synthetic, nanopores may be prepared in a variety of ways, as exemplified in the references cited above. In some embodiments a helium ion microscope may be used to drill the synthetic nanopores in a variety of materials, e.g. as disclosed by Yang et al, Nanotechnolgy, 22: 285310 (2011), which is incorporated herein by reference. A chip that supports one or more regions of a thin-film material, e.g. silicon nitride, that has been processed to be a free-standing membrane is introduced to the helium ion microscope (HIM) chamber. HIM motor controls are used to bring a free-standing membrane into the path of the ion beam while the microscope is set for low magnification. Beam parameters including focus and stigmation are adjusted at a region adjacent to the free-standing membrane, but on the solid substrate. Once the parameters have been properly fixed, the chip position is moved such that the free-standing membrane region is centered on the ion beam scan region and the beam is blanked. The HIM field of view is set to a dimension (in um) that is sufficient to contain the entire anticipated nanopore pattern and sufficient to be useful in future optical readout (i.e. dependent on optical magnification, camera resolution, etc.). The ion beam is then rastered once through the entire field of view at a pixel dwell time that results in a total ion dose sufficient to remove all or most of the membrane autofluorescence. The field of view is then set to the proper value (smaller than that used above) to perform lithographically-defined milling of either a single nanopore or an array of nanopores. The pixel dwell time of the pattern is set to result in nanopores of one or more predetermined diameters, determined through the use of a calibration sample prior to sample processing. This entire process is repeated for each desired region on a single chip and/or for each chip introduced into the HIM chamber.

In some embodiments, the solid-state substrate may be modified to generate active sites on the surface to make it more suitable for a given application. Such modifications may be of covalent or non-covalent nature. A covalent surface modification includes a silanization step where an organosilane compound binds to silanol groups on the solid surface. For instance, the alkoxy groups of an alkoxysilane are hydrolyzed to form silanol-containing species. Reaction of these silanes involves four steps. Initially, hydrolysis of the labile groups occurs. Condensation to oligomers follows. The oligomers then hydrogen bond with hydroxyl groups of the substrate. Finally, during drying or curing, a covalent linkage is formed with the substrate with concomitant loss of water. For covalent attachment organosilanes with active side groups may be employed. Such side groups consist of, but are not limited to epoxy side chain, aldehydes, isocyanates, isothiocyanates, azides or alkynes (click chemistry) to name a few. For someone skilled in the art it is obvious that multiple ways of covalently attaching a lipid bilayer to a surface to form a tethered lipid bilayer are possible. For instance, certain side groups on an organosilane may need to be activated before being capable of binding or integrating into a supported lipid bilayer. Another way of attaching a lipid bilayer to the solid surface may be achieved through affinity binding by having one affinity partner attached to the lipid moiety part of the lipid bilayer and the second affinity partner being located on the solid surface. Such affinity pairs consist of the group of, but are not limited to biotin-streptavidin, antigen-antibody and aptamers and the corresponding target molecules. In a preferred embodiment the surface modification of the solid state nanopore includes treatment with an organosilane that renders the surface hydrophilic. To form a lipid bilayer on a solid state membrane a vesicle fusion approach is the most common way of generating stable supported lipid bilayers. Lipids are dried in vacuo to remove any solvent (usually Chloroform) and the dried lipid is then rehydrated in an aqueous buffer solution such as 10 mM MES pH 6.8, 150 mM KCl, 2 mM CaCl2. Unilaminar vesicles are extruded through a 100-1000 nm filter and applied to a hydrophilic surface where they burst and form a supported lipid bilayer.

### Labeling Polymer Analytes

As mentioned above, in some embodiments, polymer analytes, such as DNA, may be labeled using “click chemistry,” e.g. using commercially available kits (such as “Click-It” from Life Technologies, Carlsbad, Calif.). Click chemistry in general refers to a synthetic process in which two molecules are linked together by a highly efficient chemical reaction, one which is essentially irreversible, in which the yield is nearly 100%, and which produces few or no reaction byproducts. More recently, the meaning has come to refer to the cyclization reaction of a substituted alkyne with a. substituted azide to form a 1,2,3-triazole bearing the two substituents. When catalyzed by copper at room temperature the reaction is known as the Huisgen cycloaddition, and it fully satisfies the requirements for click chemistry in that no other chemical functionality on the two molecules is affected during the reaction. Thus the coupling reaction has found broad application in bioconjugate chemistry, for example, in dye labeling of DNA or proteins, where many amine, hydroxy, or thiol groups may be found. The key requirement is that an alkyne group and an azide can easily be introduced into the molecules to be coupled. For example, in the coupling of a fluorescent dye to a DNA oligonucleotide, the azide group is typically introduced synthetically into the dye, while the alkyne group is incorporated into the DNA during oligonucleotide synthesis. Upon mixing in the presence of Cu+ the two components are quickly coupled to form the triazole, in this case bearing the oligonucleotide as one substituent and the dye as the other. Another more recent advance provides the alkyne component within a strained ring structure. In this case the reaction with an azide does not require the copper catalyst, being driven by release of the ring strain energy as the triazole is formed. This is better known as the copper-free click reaction. Guidance for applying click chemistry to methods of the invention may be found in the following references which are incorporated by reference: Rostovtsev V V, Green L G; Fokin, Valery V, Sharpless K B (2002). “A Stepwise Huisgen Cycloaddition Process: Copper(I)-Catalyzed Regioselective “Ligation” of Azides and Terminal Alkynes“. Angewandte Chemie International Edition 41 (14): 2596-2599. Moses J E and Moorhouse A D (2007). “The growing applications of click chemistry”, Chem. Soc. Rev. 36 (8): 1249-1262.

A combination of 1, 2, 3 or 4 nucleotides in a nucleic acid strand may be exchanged with their labeled counterpart. The various combinations of labeled nucleotides can be sequenced in parallel, e.g., labeling a source nucleic acid or DNA with combinations of 2 labeled nucleotides in addition to the four single labeled samples, which will result in a total of 10 differently labeled sample nucleic acid molecules or DNAs (G, A, T, C, GA, GT, GC, AT, AC, TC). The resulting sequence pattern may allow for a more accurate sequence alignment due to overlapping nucleotide positions in the redundant sequence read-out. Guidance for labeling polynucleotides for optically based nanopore analysis is found in the following references which are incorporated by reference: Goodman et al, U.S. Pat. No. 5,945,312; Jett et al, U.S. Pat. No. 5,405,747; Muehlegger et al, U.S. patent publication US2004/0214221; Giller et al, Nucleic Acids Research, 31(10): 2630-2635 (2003); Tasara et al, Nucleic Acids Research, 31(10): 2636-2646 (2003); Augustin et al, J. Biotechnology, 86: 289-301 (2001); Brakmann, Current Pharmacuetical Biotechnology, 5(1): 119-126 (2004); and the like.

A method for sequencing a polymer, such as a nucleic acid molecule includes providing a nanopore or pore protein (or a synthetic pore) inserted in a membrane or membrane-like structure or other substrate. An electric field is applied to the nanopore which forces the labeled nucleic acid polymer through the nanopore, while an external monochromatic or other light source may be used to illuminate the nanopore, thereby exciting donors. As, after or before labeled nucleotides of the nucleic acid pass through, exit or enter the nanopore, energy is transferred from an adjacent donor to a nucleotide acceptor-label, which results in emission of lower energy radiation. The nucleotide label radiation is then detected by a confocal microscope setup or other optical detection system or light microscopy system capable of single molecule detection known to people having ordinary skill in the art. Examples of such detection systems include but are not limited to confocal microscopy, epifluorescent microscopy and total internal reflection fluorescent (TIRF) microscopy. Other polymers (e.g., proteins and polymers other than nucleic acids) having labeled monomers may also be sequenced according to the methods described herein. In some embodiments, fluorescent labels or donor molecules are excited in a TIRF system with an evanescent wave, sometimes referred to herein as “evanescent wave excitation.” Guidance for application of TIRF systems to nanopore-based devices is found in the following references which are incorporated by reference: Soni et al, Review of Scientific Instruments, 81: 014301 (2010); and U.S. patent publication 2012/0135410.

Each acceptor labeled monomer (e.g., nucleotide) of a polymer (e.g., nucleic acid) can interact sequentially with a donor positioned next to the exit of a nanopore or channel through which the polymer is translocated. The interaction between the donor and acceptor labels may take place outside of the nanopore channel or opening, e.g., after the acceptor labeled monomer exits the nanopore. The interaction may take place within or partially within the nanopore channel or opening, e.g., while the acceptor labeled monomer passes through, or exits the nanopore.

In some embodiments, when one of the four nucleotides of a nucleic acid is labeled, the time-dependent signal arising from the single nucleotide label emission is converted into a sequence corresponding to the positions of the labeled nucleotides in the nucleic acid sequence. The process is then repeated for each of the four nucleotides in separate samples and the four partial sequences are then aligned to assemble an entire nucleic acid sequence.

In some embodiments, when multi-color labeled nucleic acid (DNA) sequences are analyzed, the energy transfer from one or more donor labels to each of the four distinct acceptor labels that may exist on a nucleic acid molecule may result in light emission at four distinct wavelengths or colors (each associated with one of the four nucleotides) which allows for a direct sequence read-out.

### Exemplary Lipophilic Donors

As mentioned above, a feature of the invention is the use of donors or FRET pairs that are constrained to and mobile within a lipid bilayer, either by complete immersion or by way of a lipophilic anchor moiety. In some embodiments, donors (referred to herein as “mobile donors”) are constrained and mobile within a lipid bilayer. Such mobile donors may be hydrophobic and immersed in the lipid portion of a lipid bilayer or mobile donors may be amphiphilic or hydrophilic and constrained to a lipid bilayer by a lipophilic anchor moiety. Exemplary donors include, but are not limited to, fluorescein dyes with lipophilic anchor moieties, rhodamine dyes with lipophilic anchor moieties, Cy3 dyes with lipophilic anchor moieties, Alexa dyes with lipophilic anchor moieties, quantum dots derivatized with lipophilic anchor moieties, nanodiamonds derivatized with lipophilic anchor moieties, and the like. A wide selection of suitable lipophilic dyes, or membrane probes, are available commercially, e.g. Molecular Probes, Inc. or Sigma-Aldrich Co.

### Translocation Speed

An obstacle associated with nanopore based sequencing approaches has been the high translocation velocity of nucleic acid through a nanopore (˜500,000-1,000,000 nucleotides/sec) which doesn't allow for direct sequence readout due to the limited bandwidth of the recording equipment. A way of slowing down the nucleic acid translocation with two different nanopore proteins was recently shown by Cherf et al. (Nat Biotechnol. 2012 Feb. 14; 30(4):344-8) and Manrao et al. (Nat Biotechnol. 2012 Mar. 25; 30(4):349-53) and are incorporated herein by reference. Both groups used a DNA polymerase to synthesize a complementary strand from a target template which resulted in the step-wise translocation of the template DNA through the nanopore. Hence, the synthesis speed of the nucleic acid polymerase (10-500 nucleotides/sec) determined the translocation speed of the DNA and since it's roughly 3-4 orders of magnitude slower than direct nucleic acid translocation the analysis of single nucleotides became feasible. However, the polymerase-aided translocation requires significant sample preparation to generate a binding site for the polymerase and the nucleic acid synthesis has to be blocked in bulk and can only start once the nucleic acid-polymerase complex is captured by the nanopore protein. This results in a rather complex set-up which might prevent the implementation in a commercial setting. Furthermore, fluctuation in polymerase synthesis reactions such as a stalled polymerization as well as the dissociation of the polymerase from the nucleic acid may hamper the sequence read-out resulting in a high error rate and reduced read-length, respectively. Optical nanopore sequencing as described in this application uses a different way of slowing down the DNA translocation. A target nucleic acid is enzymatically copied by incorporating fluorescent modified nucleotides. The resulting labeled nucleic acid has an increased nominal diameter which results in a decreased translocation velocity when pulled through a nanopore. The preferred translocation rate for optical sequencing lies in the range of 1-1000 nucleotides per second with a more preferred range of 200-800 nucleotides per second and a most preferred translocation rate of 200-600 nucleotides per second.

Alternatively, translocation speed of a polynucleotide, especially a single stranded polynucleotide, may be controlled by employing a nanopore dimensioned so that adducts and/or labels, e.g. organic dyes attached to bases, inhibit but do not prevent polynucleotide translocation. A translocation speed may be selected by attaching labels and/or adducts at a predetermined density. Such labels and/or adducts may have regular spaced attachments, e.g. every third nucleotide or the like, or they may have random, or pseudorandom attachments, e.g. every C may be labeled. In some embodiments, a selected number of different nucleotides may be labeled, e.g. every A and C, or every A and G, or every A and T, or every C, or the like, that results in an average translocation speed. Such average speed may be decreased by attaching adducts to unlabeled nucleotides. Adducts include any molecule, usually an organic molecule, that may be attached to a nucleotide using conventional chemistries. Typically adducts have a molecular weight in the same range as common organic dyes, e.g. fluorescein, Cy3, or the like. Adducts may or may not be capable of generating signals, that is, serving as a label. In some embodiments, adducts and/or labels are attached to bases of nucleotides. In other embodiments, labels and/or adducts may be attached to linkages between nucleosides in a polynucleotide. In one aspect, a method of controlling translocation velocity of a single stranded polynucleotide through a nanopore comprises the step of attaching adducts to the polynucleotide at a density, wherein translocation velocity of the single stranded polynucleotide monotonically decreases with a larger number of adducts attached, or with the density of adducts attached. In some embodiments, not every kind of nucleotide of a polynucleotide is labeled. For example, four different sets of a polynucleotide may be produced where nucleotides of each set are labeled with the same molecule, e.g. a fluorescent organic dye acceptor, but in each set a different kind of nucleotide will be labeled. Thus, in set 1 only A's may be labeled; in set 2 only C's may be labeled; in set 3 only G's may be labeled; and so on. After such labeling, the four sets of polynucleotides may then be analyzed separately in accordance with the invention and a nucleotide sequence of the polynucleotide determined from the data generated in the four analyses. In such embodiments, and similar embodiments, e.g. two labels are used, where some of the nucleotides of a polynucleotide are not labeled, translocation speed through a nanopore will be affected by the distribution of label along the polynucleotide. To prevent such variability in translocation speed, in some embodiments, nucleotides that are not labeled with an acceptor or donor for generating signals to determine nucleotide sequence, may be modified by attaching a non-signal-producing adduct that has substantially the same effect on translocation speed as the signal-producing labels.

### Nanopore Sequencing

In some embodiments, the invention is directed to the use of nanopores and fluorescence resonance energy transfer (FRET) to sequentially identify monomers of polymer analytes, such as a polynucleotide. Such analysis of polymer analytes may be carried out on single polymer analytes or on pluralities of polymer analytes in parallel at the same time, for example, using an array of nanopores.

In some embodiments, methods of determining a sequence of labeled monomers of a polymer comprise steps of: (a) providing a device comprising: (i) a solid phase membrane separating a first chamber and a second chamber, the solid phase membrane having at least one aperture connecting the first chamber and the second chamber, and the solid phase membrane having a lipid bilayer on at least one surface; (ii) a protein nanopore immobilized in the aperture, the protein nanopore having a bore, the protein nanopore contacting and extending through the lipid bilayer so that the first chamber and the second chamber are in fluid communication through the bore; and (iii) donors of one or more FRET pairs diffusably disposed in the lipid bilayer; (b) translocating the polynucleotide through the protein nanopore so that acceptor labels attached to the polynucleotide passes sequentially therethrough and so that whenever an acceptor label exits the bore of the protein nanopore at least one donor with a predetermined likelihood is within a FRET distance of such acceptor label; and (c) determining a nucleotide sequence of the polynucleotide by a sequence of FRET interactions between donors and acceptor labels exiting the bore or the protein nanopore. In some embodiments, such predetermined likelihood is a likelihood of at least ninety percent; in other embodiments, such predetermined likelihood is a likelihood of at least ninety-nine percent; in other embodiments, such predetermined likelihood is a likelihood of at least 99.9 percent.

A likelihood that a FRET interaction will occur when an acceptor exits (or enters—depending on embodiment) a nanopore depends in part on whether an excited donor happens to be within a FRET distance of the acceptor at the time of exit (or during the time interval when the acceptor move through a FRET zone (“FRET interval”)). Thus, the likelihood of a FRET interaction occurring depends on the likelihood of an excited donor being in the FRET zone or entering the FRET zone during a FRET interval of a translocating acceptor. The latter likelihood depends on the concentration and mobility of donors in the lipid bilayer. Both of these quantities are design selections of a practitioner of the invention. A higher concentration and greater mobility leads to donors entering a FRET zone more frequently; and conversely, lower concentration and lower mobility leads to donors entering a FRET zone at a lower frequency. In some embodiments, these parameters are selected so that the frequency of new (i.e. non-degraded, non-bleached) donors entering a FRET zone is equal to or greater than the frequency with which acceptors translocate the same FRET zone. In other embodiments, such as where donors and FRET zones are intermittently illuminated with alternating light and dark cycles, lower concentrations and mobilities may be selected and durations of dark cycles selected to permit replenishment of donors in FRET zones.

In other embodiments, devices and methods of the invention may use the property of protein nanopores to dampen or suppress fluorescence of translocating fluorescently labeled polymer, such as a fluorescently labeled polynucleotide. In such embodiments, monomers are labeled with fluorescent labels that are capable of at least three states while attached to a target polymer: (i) A quenched state wherein fluorescence of an attached fluorescent label is quenched by a fluorescent label on an immediately adjacent monomer; for example, a fluorescent label attached to a polymer in accordance with the invention is quenched when the labeled polymer is free in an aqueous solution. (ii) A sterically constrained state wherein a labeled polymer is translocating through a nanopore such that the free-solution movements or alignments of an attached fluorescent label is disrupted or limited so that there is little or no detectable signal generated from the fluorescent label. (iii) A transition state wherein a fluorescent label attached to a polymer transitions from the sterically constrained state to the quenched state as the fluorescent label exits the nanopore (during a “transition interval”) while the polymer translocates through the nanopore. In part, this embodiment is an application of the discovery that during the transition interval a fluorescent label is capable of generating a detectable fluorescent signal. Without the intention of being limited by any theory underlying this discovery, it is believed that the fluorescent signal generated during the transition interval is due to a freely rotatable dipole. In both, the sterically constrained state as well as the quenched state the dipoles are limited in their rotational freedom thereby reducing or limiting the number of emitted photons. In some embodiments, the translocating labeled polymer is a polynucleotide, usually a single stranded polynucleotide, such as, DNA or RNA, but especially DNA. In some embodiments, the invention includes a method for determining a nucleotide sequence of a polynucleotide by recording signals generated by attached fluorescent labels as they exit a nanopore one at a time as a polynucleotide translocates the nanopore. Upon exit, each attached fluorescent label transitions during a transition interval from a constrained state in the nanopore to a quenched state on the polynucleotide in free solution. As mentioned above, during this transition interval or period the fluorescent label is capable of emitting a detectable fluorescent signal indicative of the nucleotide it is attached to.

In some embodiments, a nucleotide sequence of a target polynucleotide is determined by carrying out four separate reactions in which copies of the target polynucleotide have each of its four different kinds of nucleotide (A, C, G and T) labeled with a single fluorescent label. In a variant of such embodiments, a nucleotide sequence of a target polynucleotide is determined by carrying out four separate reactions in which copies of the target polynucleotide have each of its four different kinds of nucleotide (A, C, G and T) labeled with one fluorescent label while at the same time the other nucleotides on the same target polynucleotide are labeled with a second fluorescent label. For example, if a first fluorescent label is attached to A's of the target polynucleotide in a first reaction, then a second fluorescent label is attached to C's, G's and T's (i.e. to the “not-A” nucleotides) of the target polynucleotides in the first reaction. Likewise, in continuance of the example, in a second reaction, the first label is attached to C's of the target polynucleotide and the second fluorescent label is attached to A's, G's and T's (i.e. to the “not-C” nucleotides) of the target polynucleotide. And so on, for nucleotides G and T.

The same labeling scheme may be expressed in terms of conventional terminology for subsets of nucleotide types; thus, in the above example, in a first reaction, a first fluorescent label is attached to A's and a second fluorescent label is attached to B's; in a second reaction, a first fluorescent label is attached to C's and a second fluorescent label is attached to D's; in a third reaction, a first fluorescent label is attached to G's and a second fluorescent label is attached to H's; and in a fourth reaction, a first fluorescent label is attached to T's and a second fluorescent label is attached to V's.

In some embodiments, a feature of the invention is the labeling of substantially all monomers of a polymer analyte with fluorescent dyes or labels that are members of a mutually quenching set. Such sets of fluorescent dyes have the following properties: (i) each member quenches fluorescence of every member (for example, by FRET or by static or contact mechanisms), and (ii) each member generates a distinct fluorescent signal when excited and when in a non-quenching state. That is, if a mutually quenching set consists of two dyes, D1 and D2, then (i) D1 is self-quenched (e.g. by contact quenching with another D1 molecule) and it is quenched by D2 (e.g. by contact quenching) and (ii) D2 is self-quenched (e.g. by contact quenching with another D2 molecule) and it is quenched by D1 (e.g. by contact quenching). Guidance for selecting fluorescent dyes or labels for mutually quenching sets may be found in the following references, which are incorporated herein by reference: Johansson, Methods in Molecular Biology, 335: 17-29 (2006); Marras et al, Nucleic Acids Research, 30: e122 (2002); and the like. Exemplary mutually quenching sets of fluorescent dyes, or labels, may be selected from rhodamine dyes, fluorescein dyes and cyanine dyes. In one embodiment, a mutually quenching set may comprise the rhodamine dye, TAMRA, and the fluorescein dye, FAM. In another embodiment, mutually quenching sets of fluorescent dyes may be formed by selecting two or more dyes from the group consisting of Oregon Green 488, Fluorescein-EX, fluorescein isothiocyanate, Rhodamine Red-X, Lissamine rhodamine B, Calcein, Fluorescein, Rhodamine, one or more BODIPY dyes, Texas Red, Oregon Green 514, and one or more Alexa Fluors. Respresentative BODIPY dyes include BODIPY FL, BODIPY R6G, BODIPY TMR, BODIPY 581/591, BODIPY TR, BODIPY 630/650 and BODIPY 650/665. Representative Alexa Fluors include Alexa Fluor 350, 405, 430, 488, 500, 514, 532, 546, 555, 568, 594, 610, 633, 635, 647, 660, 680, 700, 750 and 790.

A wide range of embodiments of the above may be implemented depending on the type of analytes being detected, the types of donors and acceptors employed, the physical arrangement of the nanopores, donors and acceptors, whether analytes are labeled with donors or with acceptors, and the like. In one embodiment, analytes measured by the invention are acceptor-labeled polymers, especially acceptor-labeled polynucleotides. In one species of the latter embodiment, different nucleotides of a polynucleotide analyte are labeled with one or more different kinds of acceptors, so that a nucleotide sequence of the polynucleotide may be determined from measuring FRET signals generated as it translocates through a nanopore. In another embodiment, analytes measured by the invention are donor-labeled polymers, especially donor-labeled polynucleotides. The sequence of the polynucleotide may be determined from measuring FRET signals as it translocates through a nanopore. In yet another embodiment of the present invention, at least one of the four nucleotides of a polynucleotide analyte is labeled with a member of a FRET pair. The positions of the labeled nucleotides in the polynucleotide are determined by translocating the labeled polynucleotide through a labeled nanopore and measuring FRET events. By labeling the remaining nucleotides of the same polynucleotide sample and subsequently translocating said samples through a labeled nanopore, sub-sequences of the polynucleotide are generated. Such sub-sequences can be re-aligned resulting in a full sequence of the polynucleotide.

In another embodiment the invention is directed to a device for analyzing one or more labeled polymer analytes, such as a device for determining a nucleotide sequence of one or more labeled polynucleotide analytes, such device comprising the following elements: (a) a solid phase membrane separating a first chamber and a second chamber, the solid phase membrane having an array of nanopores each fluidly connecting the first chamber and the second chamber through a bore or lumen, the bore or lumen having a cross-sectional dimension such that labels of a labeled polymer translocating therethrough are sterically constrained so that detectable signals are not generated, and so that the labels of adjacent monomers of the labeled polymer are self-quenching; (b) an excitation source for exciting each label when it exits each nanopore and enters the second chamber so that a signal is generated indicative of a monomer to which the label is attached; and (c) a detector for collecting at least a portion of the signal generated by each excited label; and (d) identifying the monomer to which the excited label is attached by the collected signal whenever emitted from a sequence-able nanopore; and wherein the array of nanopores is an array of clusters of nanopores.

In another embodiment, the invention is directed to a system for analyzing polymers comprising monomers that are substantially all labeled with a mutually quenching dye set and a nanopore device for sequentially detecting optical signals from the dyes of the mutually quenching dye set which are attached to the polymer. Such an embodiment for determining a sequence of a polynucleotide may comprise the following elements: (a) a solid phase membrane separating a first chamber and a second chamber, the solid phase membrane having an array of apertures each connecting the first chamber and the second chamber, and having a hydrophilic coating on at least one surface; (b) a lipid bilayer disposed on the hydrophilic coating; (c) protein nanopores immobilized in the aperture-spanning lipid bilayer, the protein nanopores each having a bore with an exit, and the protein nanopores interacting with the lipid bilayer to form a seal with the solid phase membrane in the apertures so that fluid communication between the first chamber and the second chamber occurs solely through the bore of the protein nanopore, and the protein nanopores each being cross-sectionally dimensioned so that nucleotides of the polynucleotide pass through the exit of the bore in sequence and so that fluorescent labels attached to the polynucleotide are sterically constrained; and (d) a first member of the FRET pair attached to the solid phase membrane or the protein nanopore, so that whenever nucleotides of the polynucleotide emerge from the bore, a plurality of the nucleotides are within a FRET distance of the first member of the FRET pair; and wherein the array of apertures is an array of clusters of apertures.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

### Definitions

“Evanescent field” means a non-propagating electromagnetic field; that is, it is an electromagnetic field in which the average value of the Poynting vector is zero.

“FRET” or “FOrster, or fluorescence, resonant energy transfer” means a non-radiative dipole-dipole energy transfer mechanism from a donor to acceptor fluorophore. The efficiency of FRET may be dependent upon the distance between donor and acceptor as well as the properties of the fluorophores (Stryer, L., Annu Rev Biochem. 47 (1978): 819-846). “FRET distance” means a distance between a FRET donor and a FRET acceptor over which a FRET interaction can take place and a detectable FRET signal produced by the FRET acceptor.

“Nanopore” means any opening positioned in a substrate that allows the passage of analytes through the substrate in a predetermined or discernable order, or in the case of polymer analytes, passage of their monomeric units through the substrate in a pretermined or discernible order. In the latter case, a predetermined or discernible order may be the primary sequence of monomeric units in the polymer. Examples of nanopores include proteinaceous or protein based nanopores, synthetic or solid state nanopores, and hybrid nanopores comprising a solid state nanopore having a protein nanopore embedded therein. A nanopore may have an inner diameter of 1-10 nm or 1-5 nm or 1-3 nm. Examples of protein nanopores include but are not limited to, alpha-hemolysin, voltage-dependent mitochondrial porin (VDAC), OmpF, OmpC, MspA and LamB (maltoporin), e.g. disclosed in Rhee, M. et al., Trends in Biotechnology, 25(4) (2007): 174-181; Bayley et al (cited above); Gundlach et al, U.S. patent publication 2012/0055792; and the like, which are incorporated herein by reference. Any protein pore that allows the translocation of single nucleic acid molecules may be employed. A nanopore protein may be labeled at a specific site on the exterior of the pore, or at a specific site on the exterior of one or more monomer units making up the pore forming protein. Pore proteins are chosen from a group of proteins such as, but not limited to, alpha-hemolysin, MspA, voltage-dependent mitochondrial porin (VDAC), Anthrax porin, OmpF, OmpC and LamB (maltoporin). A synthetic nanopore, or solid-state nanopore, may be created in various forms of solid substrates, examples of which include but are not limited to silicones (e.g. Si3N4, SiO2), metals, metal oxides (e.g. Al2O3) plastics, glass, semiconductor material, and combinations thereof. A synthetic nanopore may be more stable than a biological protein pore positioned in a lipid bilayer membrane. A synthetic nanopore may also be created by using a carbon nanotube embedded in a suitable substrate such as but not limited to polymerized epoxy. Carbon nanotubes can have uniform and well-defined chemical and structural properties. Various sized carbon nanotubes can be obtained, ranging from one to hundreds of nanometers. The surface charge of a carbon nanotube is known to be about zero, and as a result, electrophoretic transport of a nucleic acid through the nanopore becomes simple and predictable (Ito, T. et al., Chem. Commun. 12 (2003): 1482-83). The substrate surface of a synthetic nanopore may be chemically modified to allow for covalent attachment of the protein pore or to render the surface properties suitable for optical nanopore sequencing. Such surface modifications can be covalent or non-covalent. Most covalent modification include an organosilane deposition for which the most common protocols are described: 1) Deposition from aqueous alcohol. This is the most facile method for preparing silylated surfaces. A 95% ethanol-5% water solution is adjusted to pH 4.5-5.5 with acetic acid. Silane is added with stirring to yield a 2% final concentration. After hydrolysis and silanol group formation the substrate is added for 2-5 min. After rinsed free of excess materials by dipping briefly in ethanol. Cure of the silane layer is for 5-10 min at 110 degrees Celsius. 2) Vapor Phase Deposition. Silanes can be applied to substrates under dry aprotic conditions by chemical vapor deposition methods. These methods favor monolayer deposition. In closed chamber designs, substrates are heated to sufficient temperature to achieve 5 mm vapor pressure. Alternatively, vacuum can be applied until silane evaporation is observed. 3) Spin-on deposition. Spin-on applications can be made under hydrolytic conditions which favor maximum functionalization and polylayer deposition or dry conditions which favor monolayer deposition.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Sequence determination”, “sequencing” or “determining a nucleotide sequence” or like terms in reference to polynucleotides includes determination of partial as well as full sequence information of the polynucleotide. That is, the terms include sequences of subsets of the full set of four natural nucleotides, A, C, G and T, such as, for example, a sequence of just A's and C's of a target polynucleotide. That is, the terms include the determination of the identities, ordering, and locations of one, two, three or all of the four types of nucleotides within a target polynucleotide. In some embodiments, the terms include the determination of the identities, ordering, and locations of two, three or all of the four types of nucleotides within a target polynucleotide. In some embodiments sequence determination may be accomplished by identifying the ordering and locations of a single type of nucleotide, e.g. cytosines, within the target polynucleotide “catcgc . . . ” so that its sequence is represented as a binary code, e.g. “100101 . . . ” representing “c-(not c)(not c)c-(not c)-c . . . ” and the like. In some embodiments, the terms may also include subsequences of a target polynucleotide that serve as a fingerprint for the target polynucleotide; that is, subsequences that uniquely identify a target polynucleotide within a set of polynucleotides, e.g. all different RNA sequences expressed by a cell.

What is claimed is:

1. A device for detecting an analyte, the device comprising:
a solid phase membrane separating a first chamber and a second chamber, the solid phase membrane having at least one aperture connecting the first chamber and the second chamber
a lipid bilayer disposed on at least one surface of the solid phase membrane, the lipid bilayer comprising a concentration of donors of a fluorescence resonance energy transfer (FRET) pair, the donors being mobile within the lipid bilayer; and
at least one nanopore immobilized in the aperture, each nanopore having a bore with an entrance and an exit and the nanopore interacting with the lipid layer so that fluid communication between the first chamber and the second chamber occurs solely through one or more bores of the at least one nanopore;
wherein the concentration of donors in or on the lipid bilayer is selected so that whenever an analyte labeled with an acceptor of the FRET pair exits or enters the bore of the nanopore with a predetermined likelihood at least one donor is within a FRET distance of the acceptor.

2. The device of claim 1 wherein said nanopore is a protein nanopore.

3. The device of claim 2 wherein said concentration of donors in said lipid bilayer is selected so that an expected nearest neighbor distance between said bore of said nanopore and said at least one donor is less than or equal to said FRET distance.

4. The device of claim 1 wherein said concentration of donors is selected so that an expected frequency of donors coming within a FRET distance of said entrance or said exit of said bore is equal to or greater than a frequency with which said acceptors enter or exit said bore, respectively.

5. The device of claim 4 wherein said concentration of donors is selected so that an expected frequency of donors coming within a FRET distance of said entrance or said exit of said bore is at least ten times greater than a frequency with which said acceptors enter or exit said bore, respectively.

6. The device of claim 1 wherein said lipid bilayer is disposed on a trans surface of said solid phase membrane and said exit of said bore is located on the trans surface of said solid phase membrane and wherein said concentration of said donors is selected so that whenever said analyte labeled with said acceptor exits said bore with said predetermined likelihood at least one donor is within said FRET distance of said acceptor.

7. The device of claim 1 wherein said at least one nanopore is a single nanopore.

8. The device of claim 7 wherein said single nanopore is a protein nanopore.

9. The device of claim 1 wherein said donors in said lipid bilayer are capable of FRET only in a proximity of said at least nanopore.

10. The device of claim 9 wherein said proximity is an area encompassing a radial distance of at most 1 μm from said at least one nanopore.

11. The device of claim 9 wherein said donors in said lipid bilayer are substantially non-fluorescent outside a proximity of said at least one nanopore.

12. The device of claim 9 wherein said donors are activator-sensitive dyes that are capable of FRET only in the presence of a predetermined concentration of a chemical activator.

13. The device of claim 12 wherein said first chamber has substantially a zero concentration of chemical activator and said second chamber has a concentration of chemical activator equal to or greater than the predetermined concentration and wherein the chemical activators of the second chamber diffuse through said bores of said nanopores and create at said exit of each of said nanopores a concentration gradient in the proximity thereof.

14. The device of claim 13 where said concentration of chemical activator in said second chamber is selected so that said concentration of said chemical activator within a FRET distance of at least one of said exits renders said donors capable of FRET within said FRET distance.

15. The device of claim 14 wherein said chemical activator is a selected ion, pH, an oxidizing agent, or a reducing agent.

16. The device of claim 15 wherein said chemical activator is a selected ion and said activator-sensitive dye is an ion-sensitive dye.

17. A method of determining a nucleotide sequence of a polynucleotide, the method comprising the steps of:
(a) providing a device comprising:
(i) a solid phase membrane separating a first chamber and a second chamber, the solid phase membrane having at least one aperture connecting the first chamber and the second chamber, and the solid phase membrane having a lipid bilayer on at least one surface;
(ii) a protein nanopore immobilized in the aperture, the protein nanopore having a bore, the protein nanopore contacting and extending through the lipid bilayer so that the first chamber and the second chamber are in fluid communication through the bore; and
(iii) donors of one or more FRET pairs diffusably disposed in or on the lipid bilayer;
(b) translocating the polynucleotide through the protein nanopore so that acceptor labels attached to the polynucleotide passes sequentially therethrough and so that whenever an acceptor label exits the bore of the protein nanopore at least one donor with a predetermined likelihood is within a FRET distance of such acceptor label; and
(c) determining a nucleotide sequence of the polynucleotide by a sequence of FRET interactions between donors and acceptor labels exiting the bore or the protein nanopore.

18. The method of claim 17 wherein said donors are disposed in said lipid bilayer at a concentration selected so that a frequency of said acceptor labels exiting said bore of said protein nanopore is less than or equal to a frequency of said donors coming within a FRET distance of said exit of said bore.

19. A method of determining a nucleotide sequence of a polynucleotide, the method comprising the steps of:
translocating a polynucleotide through a protein nanopore, the polynucleotide having monomers labeled with acceptors of one or more fluorescent resonance energy transfer (FRET) pairs and the protein nanopore being immobilized in an aperture through a solid phase membrane, wherein a surface of the solid phase membrane has a lipid bilayer containing concentrations of donors movably disposed therein and the protein nanopore has a bore and contacts and extends through the lipid bilayer so that the first chamber and the second chamber are in fluid communication through the bore, and wherein the concentrations of donors are selected so that whenever a monomer of the polynucleotide having an acceptor attached traverses the bore, such acceptor passes within a FRET distance of at least one donor of its FRET pair to generate a FRET interaction; and
determining a nucleotide sequence of the polynucleotide by a sequence of FRET interactions.

20. The method of claim 19 wherein said monomers are labeled with at least two FRET pairs comprising at least two different acceptors that generate distinguishable FRET signals.

21. The method of claim 20 wherein said at least two FRET pairs comprise at least two FRET donors.

22. The method of claim 19 wherein concentrations of said donors and said translocation speeds are selected so that so that whenever said monomer labeled with an acceptor of a FRET pair exits or enters the bore of said nanopore at least one donor is within a FRET distance of the acceptor with a likelihood of at least ninety-nine percent.
